(19) United States
(12) Patent Application Publication
(10) Pub. No.: US 2017/0360559 A1
(43) Pub. Date: Dec. 21, 2017

(54) HEART VALVE PROSTHESIS WITH OUTER SKIRT

(71) Applicant: BIOTRONIK AG, Buelach (CH)

(72) Inventor: Arnulf Mayer, Buelach (CH)

(21) Appl. No.: 15/617,855

(22) Filed: Jun. 8, 2017

(30) Foreign Application Priority Data
Jun. 17, 2016 (DE) 10 2016 111 103.1

(51) Int. Cl. A61F 2/24; A61L 27/36; A61L 27/50; A61L 27/34

(52) U.S. Cl. CPC A61F 2/2418; A61L 27/34; A61L 27/3625; A61L 27/50; A61F 2/2421; A61F 2/2433; A61F 2210/0014; A61F 2210/0076; A61F 2220/0075

(57) ABSTRACT

A heart valve prosthesis includes a heart valve encircled by a base body in a peripheral direction to hold the heart valve. The heart valve is fastened to the base body and arranged in its interior. An inner skirt is arranged in the interior and is fastened to an inner surface of the base body. An outer skirt is arranged on the outer surface of the base body. The inner and outer skirts form a receptacle to accept and collect blood during diastole.

### PRIORITY CLAIM

This application claims priority under 35 U.S.C. §119 and all applicable statutes and treaties from prior German Application DE 10 2016 111 103.1, filed Jun. 17, 2016.

### FIELD OF THE INVENTION

A field of the invention is heart valve prostheses. A heart valve prosthesis of the invention serves to replace a heart valve of a patient, and is suitable for sealing paravalvular leakages.

### BACKGROUND

Heart valve prostheses are used, e.g., in transcatheter aortic valve implantations (TAVI). Paravalvular leakage, which involves blood flowing through between the vascular wall and the prosthesis, represents one of the severe complications that limits the procedural efficacy of minimally invasive heart valves.

EP 2749254A1 proposes a tissue seal that extends from a distal end of the heart valve back in the proximal direction over an anchor of the heart valve, the tissue seal tissue lying in folds in the expanded state of the prosthesis and forming tissue loops and pockets that can hold blood, and thus can have a sealing effect. However, this arrangement is not capable of filling with blood in a targeted and effective manner, especially in diastole, to close paravalvular leakages when the artificial heart valve is closed.

### SUMMARY OF THE INVENTION

The invention relates to a heart valve prosthesis that counteracts the risk of paravalvular leakage and provides targeted and improved sealing. A preferred embodiment provides a heart valve prosthesis that includes a heart valve encircled by a base body in a peripheral direction to hold the heart valve. The heart valve is fastened to the base body and arranged in its interior. An inner skirt is arranged in the interior and is fastened to an inner surface of the base body. An outer skirt is arranged on the outer surface of the base body. The inner and outer skirts form a receptacle to accept and collect blood during diastole.

### BRIEF DESCRIPTION OF THE DRAWINGS

Advantages and expedient features of the invention will also become clear from the following description of an exemplary embodiment provided with reference to the FIGURES, in which:

FIG. 1 is a schematic, partly cutaway view of preferred embodiment heart valve prosthesis.

### DETAILED DESCRIPTION OF THE PREFERRED EMBODIMENTS

To avoid paravalvular leakage of the heart valve prosthesis, the preferred embodiment heart valve prostheses additionally have a (preferably separate) outer skirt arranged on the outer surface of the base body, this outer skirt surrounding the base body (preferably the outer skirt surrounds the inflow area of the base body on the outside, so that the corresponding section of the base body is arranged between the inner skirt and the outer skirt) and circling in the peripheral direction, the two skirts forming a receptacle to accept blood, this receptacle being configured to accept and collect blood during diastole, so that the outer skirt permanently bulges in the direction of the patient's tissue surrounding the heart valve prosthesis, and lies against it to seal it. This effectively suppresses paravalvular leakage, or significantly reduces such a risk.

Preferably, the aforementioned tissue is the anulus of a native aortic valve (one that is defective or to be replaced), or at least a section of the left ventricular outflow tract (LVOT). That is, the sealing preferably takes place on the anulus or the LVOT.

A preferred heart valve prosthesis has a heart valve that is intended to replace a heart valve of the patient, and a base body, preferably in the form of a stent, that serves to hold the replacement heart valve, the replacement heart valve being fastened to the base body. The heart valve prosthesis also comprises a skirt arrangement, which is suitable for sealing paravalvular leakages.

A preferred embodiment of the inventive heart valve prosthesis provides that the base body extends in an axial direction starting from an inflow area, through which the blood can flow in a direction of flow or in the axial direction into the interior of the base body, toward an outflow area, through which blood passing through the heart valve can exit from the interior of the base body in the direction of flow or in the axial direction.

A preferred embodiment of the inventive heart valve prosthesis further provides that the outer skirt can have a first (in particular circling) edge area and a second (in particular circling) edge area, the two edge areas being connected together (e.g., in a single piece) through a middle section of the outer skirt.

A particularly preferred embodiment provides that the first edge area of the outer skirt be fastened, in particular sewn on, to an edge area of the base body bordering the inflow area of the base body. Here it is especially preferred that the first edge area of the outer skirt be fastened, preferably sewn on, to the base body in the inflow area, so that the base body is sealed off, in particular sealed off to flow, with the inner skirt fastened inside in the inflow area. This advantageously ensures that during the diastole blood can collect in the area between the outer skirt and the inner skirt, and that the amount of collected blood increases with every diastole until the area between them is under pressure and amply filled with blood.

Relating to this, an embodiment preferably further provides that the middle section of the outer skirt not be fastened to the base body, or be fastened to it only loosely, so that the middle section can bulge toward the tissue. In particular, this applies both when the base body is in the compressed state (for example when it is being transported to the implantation site by means of a catheter), and also when it is in the expanded and implanted state.

A preferred embodiment further provides that the second edge area of the outer skirt be fastened, in particular sewn on, to the outflow area of the base body, or at least be fastened, in particular sewn on, to the base body adjacent to the outflow area.

An embodiment of the inventive heart valve prosthesis further provides that the inner skirt have a first (preferably circling) edge area and a second (preferably circling) edge area that is opposite the first edge area in the axial direction, the first edge area of the inner skirt preferably being fastened, in particular sewn on, to the edge area bordering the inflow area of the base body, and the second edge area of the inner skirt preferably being fastened to the base body closer to the outflow area.

An embodiment preferably further provides that the second edge area of the outer skirt be lengthened and/or project beyond the second edge area of the inner skirt in the axial direction, the second edge area of the outer skirt preferably not projecting beyond the commissures of the heart valve in the axial direction. Such an arrangement is explicitly preferred in the expanded state, when the heart valve prosthesis is implanted.

In this way, according to an embodiment of the inventive heart valve prosthesis the second edge area of the outer skirt and the second edge area of the inner skirt form an inflow opening of the aforementioned receptacle, this inflow opening circling in the peripheral direction and facing the interior of the base body. Thus, during the respective diastole, it is easy for blood to flow into the receptacle through the inflow opening and collect there, heart beat by heart beat, and the outer skirt begins to exert a sealing effect against the tissue, the outer skirt pressing more and more strongly against the surrounding anulus or LVOT as the receptacle fills. The arrangement of the outer skirt with respect to the inner skirt enlarges the cross section of the circling inflow opening preferably during diastole, which favors an inflow of blood into the receptacle.

In a preferred embodiment, the inflow opening is the arranged directly above the artificial valve leaflet. Here “above the valve leaflet” is intended to mean that the inflow opening is arranged behind the valve leaflets in the direction of blood flow. The dimensions of the base body are designed so that the valve prosthesis comes to lie in the old natural heart valve in such a way that the valve leaflets of the heart prosthesis are located in an area of the blood vessel that has a very small diameter and is very narrow in comparison with the surrounding tissue. In the case of a heart valve prosthesis for the aortic valve (an aortic heart valve prosthesis), the inflow opening is arranged in the area of the sinuses, which makes the inflow opening very wide. This is advantageous, since in the case of blood regurgitation, for example during diastole in the case of an aortic heart valve prosthesis, the blood flow is directed through the heart valve leaflets and the blood flows into the wide inflow opening with increased pressure, which causes the outer skirt to bulge with the greatest force and high blood volume. This prevents paravalvular leakage with the highest effectiveness. Pressing blood in under increased pressure also supports the formation of stationary thrombi, which form in the space between the inner and outer skirt and seal paravalvular leakage in a lasting and definitive manner.

It is preferred for the aforementioned base body to be a so-called stent, which has multiple struts that form a cell structure. This structure is characterized by multiple cells, which can be so-called open cells or closed cells.

Preferably it is further provided that the base body of the prosthesis be configured to be expanded (e.g., by a balloon in a way known in the art), in particular to expand the heart valve prosthesis into the state in which it is supposed to be at the implantation site.

An alternative embodiment of the invention provides that the base body be designed to be self-expanding. Here the base body is preferably made of a material that allows automatic expansion of the base body, as soon as it is appropriately released (e.g., due to an elastic, especially superelastic property of the base body).

It is especially preferred for the heart valve prosthesis to be configured to be implanted into a body lumen of a patient via catheter, preferably by means of TAVI. Accordingly, another aspect of the invention relates to a system with an inventive heart valve prosthesis and a catheter device, which is configured to implant the heart valve prosthesis.

An embodiment of the invention further provides that the heart valve have a biological tissue, in particular pericardium.

An embodiment of the inventive heart valve prosthesis further provides that the artificial heart valve, the outer skirt, and/or the [inner] skirt have one of the following materials or consist of one of the following materials: microbial cellulose, or further in principle all types of animal tissue, in particular tissue from mammals, including humans. Non-human tissue is preferred. In particular, tissue that can be used as valve material in a heart valve is suitable. Tissues that are preferred are pericardial tissue and heart valves, but also skin, ligament tissue, tendons, peritoneum, dura mater, submucosa, in particular of the gastrointestinal tract, or costal pleura. If the tissue used is itself a heart valve, it is possible to use all valves, that is aortic valves, pulmonary valves, mitral valves, and tricuspid valves. In addition, pericardial tissue from pigs, sheep, goats, horses, crocodiles, kangaroos, ostriches, and cattle is preferred. The heart valve, inner skirt, and outer skirt can be made of the same material or of different materials.

Furthermore, the outer and inner skirt can, in particular, also be made of a synthetic polymer material, or comprise such a material. In a preferred embodiment, the outer and/or inner skirt are made of polyethers, polyolefins, especially polyethylene, polypropylene, and polytetrafluoroethylene, or polyesters, especially Dacron®. In a special embodiment, the outer skirt is made of one of the above-mentioned synthetic polymer materials, especially polytetrafluoroethylene or Dacron®. In another embodiment, the outer skirt can be made of a pliable or elastic material that makes it possible to improve the lining, and thus sealing of areas in which paravalvular leakage occurs, by increased pressure in the receptacle between the outer and inner skirt.

It is especially preferable to provide that the heart valve be designed to replace a defective native aortic valve.

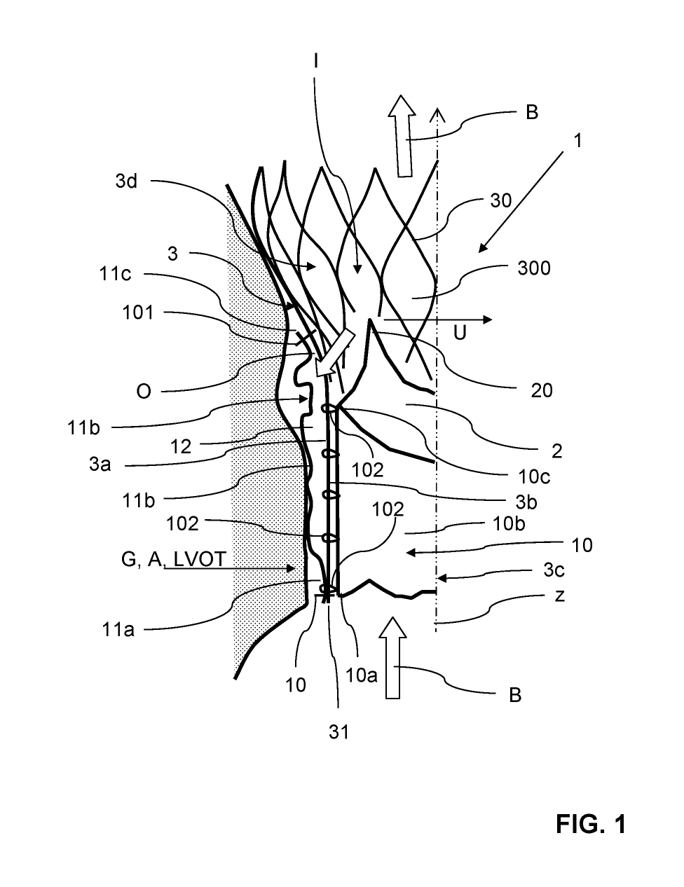

FIG. 1 shows the heart valve prosthesis 1, which has a preferably artificial heart valve 2 made of a biological tissue with commissures 20, and a base body 3, preferably in the form of an expandable or self-expanding stent (also see above) that is configured to hold the heart valve 2 in an interior I surrounded by the base body 3, the heart valve 2 being fastened to the base body 3, e.g., by sewing the heart valve 2 with the base body 3. The base body 3 preferably has multiple struts 30, which form a cell structure with, e.g., closed cells 300 . For clarity, FIG. 1 only shows the struts 30 in some areas, so that the skirt 10 is not concealed.

In the implanted and expanded state, the heart valve prosthesis 1 can be configured, e.g., to replace or to displace a defective aortic valve of a patient. The blood then flows in the direction of flow B through the replacement heart valve 2, which is fastened to the expanded base body 3, the prosthesis 1 being intended to lie in a circle against the surrounding tissue G or the surrounding vascular wall G, so as to seal it, this sealing preferably occurring in the area of the anulus A of the defective native heart valve or on the left ventricular outflow tract LVOT, to avoid blood flow between the tissue or the vascular wall G and the prosthesis 1. Such blood flow represents a so-called paravalvular leakage.

To prevent such paravalvular leakage, the inventive heart valve prosthesis 1 now has, arranged in the interior I, an inner skirt 10, which is fastened to an inner surface 3b of the base body 3 and circles in the peripheral direction U of the base body 3, and furthermore an outer skirt 11 arranged on the outer surface 3a of the base body 3, which surrounds the base body 3 and circles in the peripheral direction U, the two skirts 10, 11 forming a receptacle 12 to hold blood, this receptacle 12 being configured to accept and collect blood during diastole, so that the outer skirt 11 bulges in the direction of the patient's tissue G surrounding the heart valve prosthesis 1 or in the direction of the anulus A or the LVOT, and lies against these vascular structures to seal them. Thus, the receptacle 12 represents the space between the outer and inner skirt running in the axial and peripheral direction U, which is limited by the solid connection of the two skirts in the inflow area.

In particular, here it is especially provided that the base body 3 extends in an axial direction z starting from an inflow area 3c of the base body 3 or the prosthesis 1, through which blood can flow in a direction of flow B into the interior I, toward an outflow area 3d of the base body 3 or the prosthesis 1, through which blood passing through the heart valve 2 can exit from the interior (I) in the direction of flow B.

According to FIG. 1, the outer skirt 11 has a first edge area 11a circling in the peripheral direction U and a second edge area 11c circling in the peripheral direction U, the two edge areas 11a, 11c being connected through a middle section 11b of the outer skirt 11, for example, and preferably connected together in a single piece. Here the first edge area 11 a of the outer skirt 11 is fastened, e.g., by means of suitable sutures 100 or another suitable means of connection 100, to an edge area 31 of the base body 3, this edge area 31 bordering the inflow area 3c. Furthermore, the second edge area 11c of the outer skirt 11 is fastened, e.g., also by means of suitable sutures 101 or another suitable means of connection 101, to the outflow area 3d of the base body 3. In contrast to the two edge areas 11a, 11c, the middle section 11b of the outer skirt is not fastened to the base body 3, or only loosely fastened to it, so that the base body 3 forms a loose material layer, which can bulge toward the outside for sealing purposes, if the receptacle 12 fills with blood.

On the inner surface 3a of the base body 3, the receptacle 12 is bordered by the inner skirt 10, which has, one after the other in the axial direction z, a first edge area 10a, then a middle section 10b, and after that a second edge area 10c, wherein 10b is lining the inflow area of the base body 3 or the prosthesis 1.

The first edge area 10a of the inner skirt 10 is again fastened to the edge area 31 bordering the inflow area 3c of the base body 3, for which purpose, e.g., sutures 102 or another suitable means of connection 102 can be used. By contrast, the second edge area 10c of the inner skirt 10 is located closer to the outflow area 3d, where it is fastened to the base body 3, e.g., by means of sutures 102 or another suitable means of connection 102.

It is especially preferred for it to be provided that the second edge area 11c of the outer skirt 11 extend in the axial direction z beyond the second edge area 10c of the inner skirt 10. Furthermore, it is preferred that the second edge area 11c of the outer skirt 11 not extend in the axial direction z beyond the commissures 20 of the heart valve 2. Such an arrangement is explicitly present in the expanded state, when the heart valve prosthesis is implanted.

In this way, the second edge area 11c of the outer skirt 11 and the second edge area 10c of the inner skirt 10 form an inflow opening O of the receptacle 12, this inflow opening O circling in the peripheral direction U and allowing blood to get into the receptacle 12 in diastole and collect there, which causes the sealing effect of the outer skirt to begin.

1. A heart valve prosthesis comprising:
a heart valve,
a base body encircling the heart valve in a peripheral direction to hold the heart valve, the heart valve being fastened to the base body and arranged in its interior,
an inner skirt arranged in the interior, the inner skirt being fastened to an inner surface of the base body and circling in the peripheral direction of the base body, and
an outer skirt arranged on the outer surface of the base body, the outer skirt surrounding the base body and circling in the peripheral direction, the inner and outer skirts being configured to form a receptacle to accept and collect blood during diastole.

2. A heart valve prosthesis according to claim 1, the outer skirt being configured to bulge in the direction of tissue to seal against it, the tissue being formed by the anulus of a native aortic valve or at least a section of the left ventricular outflow tract.

3. A heart valve prosthesis according to claim 1, wherein the outer skirt has a first edge area and a second edge area that are connected through a middle section of the outer skirt.

4. A heart valve prosthesis according to claim 3, wherein the first edge area is fastened to an edge area of the base body that borders an inflow area of the base body.

5. A heart valve prosthesis according to claim 3, wherein a middle section of the outer skirt is not fastened to the base body.

6. A heart valve prosthesis according to claim 3, wherein a middle section of the outer skirt is loosely fastened to the base body.

7. A heart valve prosthesis according to claim 3, wherein a second edge area of the outer skirt is fastened to an outflow area of the base body.

8. A heart valve prosthesis according to claim 3, the inner skirt comprising a first edge area and a second edge area following the first edge area in an axial direction, the second edge area of the outer skirt projecting beyond the second edge area of the inner skirt in the axial direction.

9. A heart valve prosthesis according to claim 8, wherein the second edge area of the outer skirt and the second edge area of the inner skirt form an inflow opening circling in the peripheral direction.

10. A heart valve prosthesis according to claim 1, wherein the base body is expandable or self-expanding.

11. A heart valve prosthesis according to claim 1, wherein the heart valve prosthesis is configured to be implantable via catheter.

12. A heart valve prosthesis according to claim 1, wherein the heart valve comprises biological tissue.

13. A heart valve prosthesis according to claim 12, wherein the biological tissue comprises pericardium.

14. A heart valve prosthesis according to claim 1, wherein the outer skirt comprises one of a synthetic polymer material, microbial cellulose, or biological tissue.

15. A heart valve prosthesis according to claim 1, wherein the outer skirt consists of one of a synthetic polymer material, microbial cellulose, or biological tissue.

16. A heart valve prosthesis according to claim 1, wherein the outer skirt comprises one of microbial cellulose or biological tissue.

17. A heart valve prosthesis according to claim 1, wherein the outer skirt consists of one of microbial cellulose or biological tissue.
